(19) United States
(12) Patent Application Publication
(10) Pub. No.: US 2012/0088953 A1
(43) Pub. Date: Apr. 12, 2012

(54) Fractured Bone Treatment Methods And Fractured Bone Treatment Assemblies

(76) Inventor: Jerry King, Missoula, MT (US)

(21) Appl. No.: 12/901,167

(22) Filed: Oct. 8, 2010

(51) Int. Cl. A61N 2/08; A61B 17/00

(52) U.S. Cl. 600/9; 606/54

(57) ABSTRACT

Fractured bone treatment methods and fractured bone treatment assemblies are described. According to one aspect, a fractured bone treatment method includes generating an attractive magnetic force proximate to a fracture in a fractured bone and maintaining pieces of the fractured bone in proper anatomical positions with respect to one another using the attractive magnetic force.

### TECHNICAL FIELD

This disclosure relates to fractured bone treatment methods and fractured bone treatment assemblies.

### BACKGROUND OF THE DISCLOSURE

Fractured bones are unfortunate medical conditions where the continuity of a bone is broken. Some individuals may be more susceptible to bone fractures than other individuals due to other medical conditions, such as cancer or osteoporosis, which may weaken the individual's bones. Other bone fractures may result from stress or high force impacts.

Humans may experience different types of bone fractures which may be treated differently. For example, closed or simple fractures include fractures where the skin is intact while open or compound fractures include fractures with associated wounds and which have associated increased risks of infection. Bone fractures may be additionally characterized by displacement and angulation and reduction of the bone fracture may be implemented to align the fractured bone if displacement or angulation of the fractured bone is relatively large. Reduction of the fractured bone is frequently implemented in surgery where the fractured bone may be reduced for proper alignment and subsequently stabilized to promote growth of bone tissue.

At least some aspects of the disclosure are directed to apparatus and methods for treating fractured bones.

### BRIEF DESCRIPTION OF THE DRAWINGS

Exemplary embodiments of the disclosure are described below with reference to the following accompanying drawings.

FIG. 1 is a side view of a fractured bone treatment assembly according to one embodiment of the disclosure.

FIG. 1A is an end view of a fractured bone treatment assembly according to one embodiment of the disclosure.

FIG. 2 is a functional block diagram of a fractured bone treatment assembly comprising one or more electromagnets according to one embodiment of the disclosure.

FIG. 3A is a side view of a separation assembly according to one embodiment of the disclosure.

FIG. 3B is a top view of a separation assembly according to one embodiment of the disclosure.

FIG. 4A is an isometric view of a magnet of a fractured bone treatment assembly according to one embodiment of the disclosure.

FIG. 4B is an end view of a plurality of magnets of a fractured bone treatment assembly according to one embodiment of the disclosure.

FIG. 5 is a cross-sectional view of a fractured bone treatment assembly comprising a carrier apparatus and a plurality of magnets according to one embodiment of the disclosure.

### DETAILED DESCRIPTION OF THE DISCLOSURE

This disclosure is submitted in furtherance of the constitutional purposes of the U.S. Patent Laws “to promote the progress of science and useful arts” (Article 1, Section 8).

According to one embodiment, a fractured bone treatment method comprises generating an attractive magnetic force proximate to a fracture in a fractured bone and maintaining pieces of the fractured bone in proper anatomical positions with respect to one another using the attractive magnetic force.

According to an additional embodiment, a fractured bone treatment method comprises providing a plurality of magnets adjacent to an exterior surface of a fractured bone at a location which is proximate to a fracture in the fractured bone, wherein the providing comprises providing the magnets in an arrangement wherein an attractive magnetic force is generated using the plurality of magnets and aligning the fractured bone using the attractive magnetic force generated the plurality of magnets.

According to another embodiment, fractured bone treatment assembly comprises a plurality of pairs of magnets, wherein the magnets of an individual one of the pairs are coupled with one another using a connecting member which is configured to permit the magnets of the individual one of the pairs to be placed adjacent to opposing exterior surfaces of a fractured bone in an arrangement wherein an attractive magnetic force is generated by the magnets which maintains the fractured bone in proper anatomical alignment and a separation assembly configured to provide separation of the magnets of the pairs from one another during placement of the magnets adjacent to the opposing exterior surfaces of the fractured bone.

At least some embodiments of the disclosure are directed towards apparatus and methods for treating fractured bones using magnetic forces. Some embodiments of the disclosure are directed towards reducing bone fractures to provide fractured bones in anatomically correct positions for bone tissue growth. Other embodiments of the disclosure are directed towards maintaining alignment of fractured bones for healing including the growth of bone tissue.

Some of the disclosed treatment methods are implemented during surgery (e.g., treatment for displaced bone fractures) and magnets may be placed within the individual while additional treatment methods are implemented without surgery, for example, by placing magnets around the exterior of an appendage or limb of the patient(e.g., treatment for non-displaced bone fractures). Different types of magnets may be used and include permanent magnets or electromagnets and the magnets may additionally have different sizes, shapes and magnetic powers, for example. Additional embodiments of the disclosure are disclosed below.

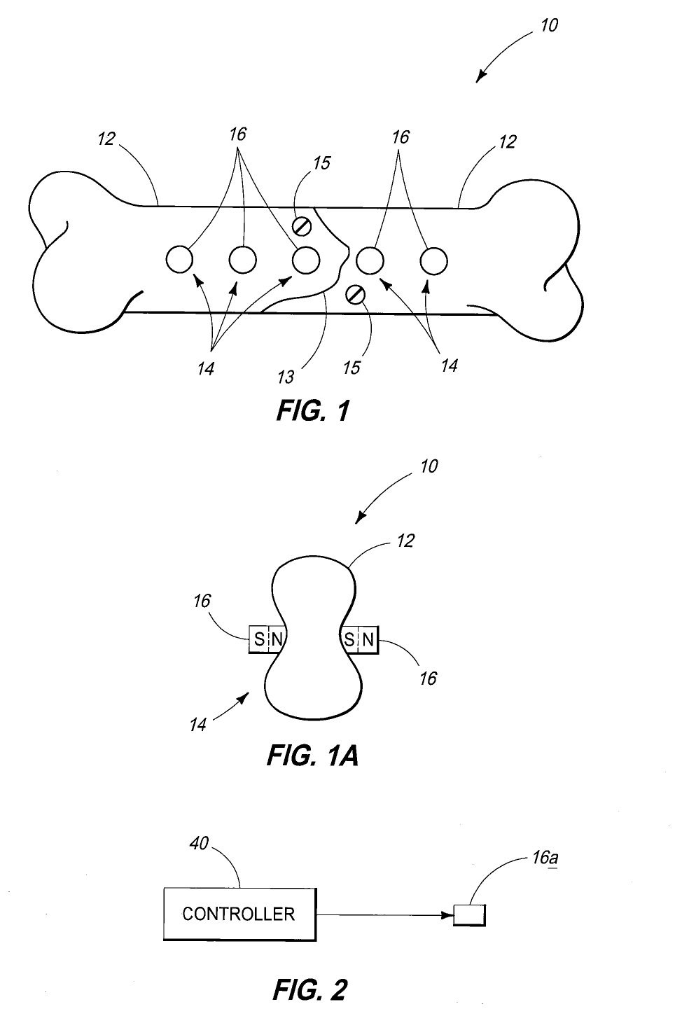

Referring to FIG. 1, one embodiment of a fractured bone treatment assembly 10 is shown for treating a fracture 13 of a fractured bone 12. In the depicted example of FIG. 1, surgery was deemed to be needed to treat the patient's fractured bone 12 and the surgeon makes an incision appropriate for the fracture 13 to be treated and the soft tissue (not shown) about the fracture 13 may be moved and held away using tissue spreading and holding tools (e.g., surgical retractors, ratchets, etc.) to expose the fractured bone 12 in one treatment example.

Different types of fractures may be treated using embodiments of the disclosure including fractures where pieces of the fractured bone 12 may remain connected to one another, or a plurality of pieces may be separated from one another or fragments may be present. At least some aspects of the disclosure refer to reducing a fractured bone (i.e., increasing alignment of pieces or fragments of the fractured bone) and/or maintaining the alignment of the pieces or fragments of the fractured bone using attractive magnetic forces. The assembly may be used alone to treat fractured bones 12 or in combination with other medical devices.

In one embodiment, the fractured bone treatment assembly 10 is initially utilized to reduce a fracture (i.e., improve the alignment of the fractured bone 12) and/or hold the fractured bone 12 in an appropriate alignment as determined by the surgeon such that additional fixing devices (e.g., screws, plates, etc.) 15 may be attached to the fractured bone 12 to treat and stabilize the fracture 13 for subsequent healing.

For example, the surgeon may utilize the fractured bone treatment assembly 10 to reduce the fractured bone 12 into a desired alignment and/or utilize the assembly 10 to hold the fractured bone 12 in the desired alignment while the additional fixing devices 15 are attached to treat the fractured bone 12. After the additional fixing devices 15 are attached to the fractured bone 12, the magnets 16 of the treatment assembly 10 may be left in place in the patient or removed from the fractured bone 12. In other embodiments, the magnets 16 are used to maintain the alignment of the fractured bone 12 for healing (e.g., including bone tissue growth) without the use of additional fixing devices 15.

The example disclosed fractured bone treatment assembly 10 of FIG. 1 is shown with respect to one side of a fractured bone 12. The illustrated assembly 10 includes one or more pairs 14 of magnets 16 which are provided adjacent to fracture 13 in the fractured bone 12 (e.g., a plurality of pairs 14 of magnets 16 are shown in the example embodiment of FIG. 1). In one embodiment, the magnets 16 of an individual pair 14 are provided opposite one another on opposing sides of the fractured bone 12. Accordingly, the magnets 16 shown in FIG. 1 are each one magnet 16 of one of five pairs 14 while the other magnets 16 of the pairs 14 are placed adjacent to the opposing side of the fracture bone 12 which is not shown in FIG. 1.

In addition, opposing poles of the magnets 16 of the individual pairs 16 are provided adjacent to the fractured bone 12 to cause the generation of an attractive magnetic force by the pair 14 of magnets 16 which maintains the magnets 16 of the pair 14 in fixed positions with respect to one another and the bone 12 as well as aligning the fractured bone 12.

For example, referring to FIG. 1A, one pair 14 of magnets 16 is shown provided about a fractured bone 12. In the illustrated arrangement, opposite North and South poles of the magnets 16 of the pair 14 are provided adjacent to the opposing sides of the fractured bone 12 to create an attractive magnetic force between the magnets 16 of the pair 14. More specifically, one of the magnets 16 of the pair 14 is provided having a North (N) pole positioned adjacent to the surface of the fractured bone 12 while the other of the magnets 16 of the pair 14 is provided having a South (S) pole positioned adjacent to the surface of the fractured bone 12 providing an attractive magnetic force between the magnets 16 of the pair 14 and which maintains the magnets 16 of the pair 14 in fixed positions with respect to one another and the fractured bone 12 as well as providing alignment of the fractured bone 12.

Different configurations of magnets 16 may be used in different embodiments. In one arrangement, the magnets 16 include permanent magnets. In one more specific embodiment, the magnets 16 are rare earth magnets, such as neodymium magnets. As discussed further below, magnets 16 may also include electromagnets.

The example embodiment described above is discussed with respect to using the attractive magnetic force to reduce a fracture of a fractured bone 12 and/or hold or maintain a fractured bone 12 in a desired alignment such that additional fixing devices 15 may be inserted or attached to the fractured bone 12 to stabilize the fractured bone 12 in the proper alignment for subsequent healing, and the magnets 16 may or may not be removed after attachment of the additional fixing devices 15.

In another embodiment, the magnets 16 are used to reduce the fracture and/or maintain the fractured bone in proper anatomical alignment for healing without additional fixing devices. More specifically, the magnets 16 may be configured to generate sufficient attractive magnetic forces to maintain the fractured bone 12 in a proper anatomical alignment. The proper anatomical alignment of the fractured bone 12 refers to acceptable alignment of the fractured bone which is suitable for healing, for example including tissue growth.

As mentioned above, the fractured bone treatment assembly 10 of FIG. 1 may also be utilized to assist the surgeon or other medical personnel with reducing the fractured bone 12 (i.e., improving the anatomical alignment of the pieces or fragments of the fractured bone) in one embodiment. For example, the magnets 16 may be implemented as electromagnets in one embodiment and placed on opposing sides of the fracture of the fractured bone 12 prior to applying electrical current to the electromagnets. The current may thereafter be increased which generates an attractive magnetic force which may reduce the fracture and improve the anatomical alignment of the fractured bone 12. In another embodiment, permanent magnets 16 are positioned about the sides of the fractured bone 12 adjacent to the location of the fracture to reduce the fracture and align the fractured bone.

Following appropriate reduction of the fracture and/or stabilizing of the fractured bone 12 for subsequent healing, the displaced soft tissue may be replaced about the fractured bone 12, magnets 16 and fixing devices 15 (if provided) and the incision may be closed following appropriate placement of the magnets 16 and/or fixing devices 15 about the fractured bone 12, alignment of the fractured bone 12 and stabilization to maintain the alignment of the fractured bone 12. The attractive magnetic forces of the magnets 16 may be used to maintain alignment of the fractured bone 12 to heal the bone including growing bone tissue in one embodiment. The magnets 16 may or may not be removed from the fractured bone 12 of the patient following passage of a sufficient time period for the bone to heal.

Referring to FIG. 2, one embodiment of an arrangement of a fractured bone treatment assembly which uses electromagnets 16a is shown. The magnitude of the magnetic force of electromagnets 16a may be controlled using a controller 40. In one embodiment, controller 40 may vary a current applied to electromagnets 16a which controls the magnitude of the magnetic force generated by the electromagnets 16a.

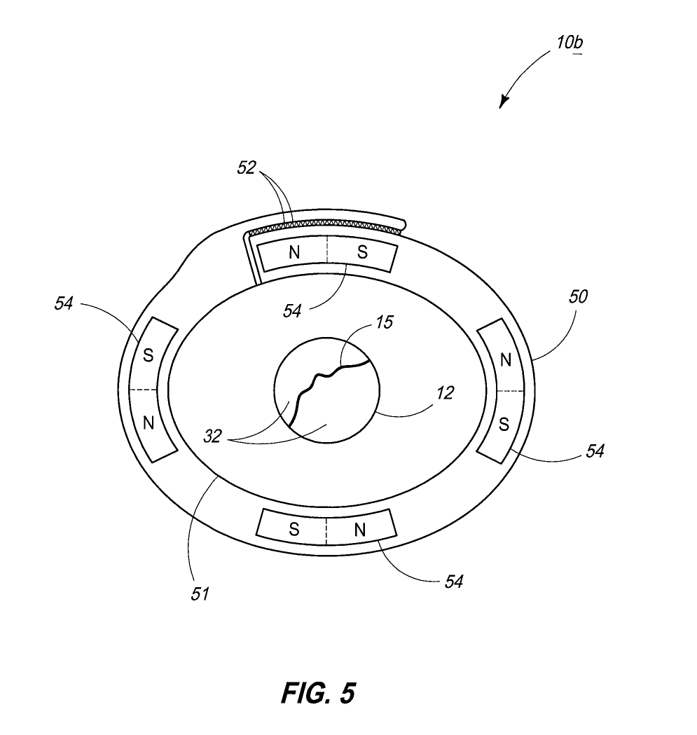

In one example, the electromagnets 16a are de-energized to enable the surgeon or other medical personnel to appropriately position the electromagnets 16a about the fractured bone 12 or appendage of the patient without attractive or repulsive interaction between the electromagnets 16a. Following appropriate positioning of one or more pairs of electromagnets 16a about the fractured bone 12 or the appendage (e.g., according to FIGS. 1 and 1A or FIG. 5 below), the controller 40 may provide current to the electromagnets 16a which causes a pair of the electromagnets 16a about the fractured bone 12 to attract to one another and attach to the fractured bone 12 and provide and/or maintain the fractured bone 12 in a desired alignment. The electromagnets 16a may be positioned internally (e.g., FIG. 1) or externally (e.g., FIG. 5) of a limb in different embodiments.

In one example, following appropriate positioning of pairs of the electromagnets 16a about opposing sides of the fracture 13, the current to the electromagnets 16a may be increased to assist with alignment of the fractured bone 12. For example, the increasing current increases the attractive magnetic force which may increase the alignment of the fractured bone 12 (i.e., reduce the fracture).

In one embodiment, the current may be maintained to the electromagnets 16a to maintain the attractive magnetic force between the pairs of electromagnets 16a and to maintain the alignment of the fractured bone 12 while additional fixing devices 15 may be attached to or inserted into the fractured bone 12. Following the attachment or insertion of the additional fixing devices 15, electromagnets 16a may remain energized or, alternatively, the electrical current may be removed, thereby de-energizing the electromagnets 16a and permitting removal of the magnets 16a of the fractured bone treatment assembly 10.

In another example, the fractured bone 12 may have been previously aligned by the surgeon (if necessary for proper treatment), and the electromagnets 16a of the treatment assembly may be de-energized and properly positioned about the fractured bone 12. Once the electromagnets 16a are appropriately positioned, current may be applied to energize the electromagnets 16a and to hold the fractured bone 12 in appropriate alignment for subsequent healing and/or the attachment of additional fixing devices 15. The electromagnets 16a may be de-energized and removed from the fractured bone 12 after the bone 12 has sufficiently healed or the additional fixing devices 15 are attached or installed in one embodiment.

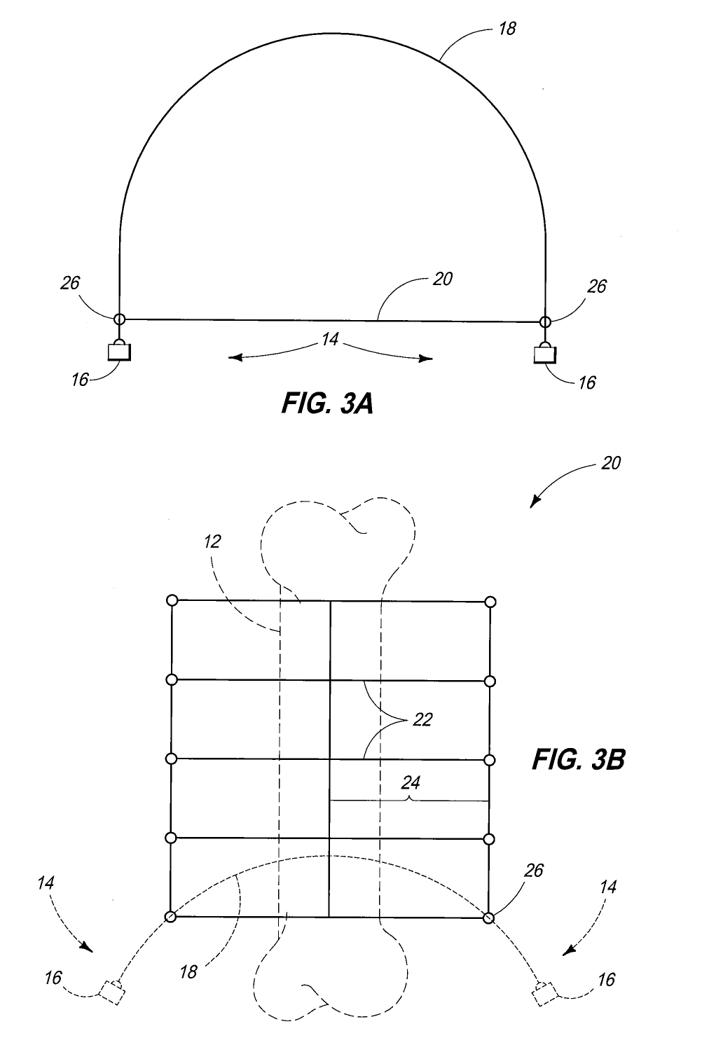

Referring to FIGS. 3A-3B, a separation assembly 20 is disclosed according to one embodiment. The separation assembly 20 is configured to assist a surgeon or other medical personnel with use of pairs 16 of magnets 14 to treat fractured bones 12. In one embodiment, separation assembly 20 is utilized with permanent magnets which may not be selectively energized or de-energized. Permanent magnets used according to some of the embodiments of the disclosure have relatively significant magnetically attractive (or repulsive) forces and separation of magnets 16 which are permitted to contact one another may be difficult. The example embodiment of the separation assembly 20 is utilized to assist the treating personnel with the provision of the pairs 14 of magnets 16 (e.g., implemented as permanent magnets) about the fractured bone 12 while maintaining separation of the magnets 16.

In the described embodiment, the magnets 16 of a pair 14 may be attached to one another by a flexible connecting member (e.g., stainless steel wire or cable or non-metallic wire or cable) 18 as shown in FIG. 3A. Opposing poles of the magnets 16 of the pair 14 may be attached to the connecting member in one embodiment (i.e., one of the magnets 16 of the pair 14 may be attached to the connecting member 18 at its North pole and the other of the magnets 16 of the pair 14 may be attached to the connecting member 18 at its South pole).

Referring to FIG. 3B, the example embodiment of the separation assembly 20 includes a plurality of horizontal and vertical non-magnetic rigid members 22, 24 which may be arranged in a cage. A plurality of non-magnetic grommets 26 may be affixed to the rigid members 22, 24 and the connecting member 18 of the pair 14 of magnets 16 may pass through respective ones of the grommets 26 in one embodiment. In the example arrangement of FIG. 3B, five pairs 14 of magnets 16 (one pair 14 is shown in FIG. 3B in phantom) may be separated using separation assembly 20 and the connecting member 18 of the pair 14 of magnets 16 passes through a pair of horizontally aligned grommets 26 of the separation assembly 20.

During use and following the incision and removal of soft tissue about the fractured bone 12, the separation assembly 20 may be provided adjacent to the exposed fractured bone 12 with the connecting members 18 of the pairs 14 of magnets 16 drawn through the side of the separation assembly 20 which is opposite to the magnets 16 to maintain separation of the magnets 16 from one another (e.g., as shown in FIG. 3A). Following appropriate placement of the separation assembly 20 adjacent to the fractured bone 12, the connecting members 18 may be drawn through the grommets 26 of the separation assembly 20 allowing the pairs of magnets 16 to attract to one another at different longitudinal locations on opposing sides of the fractured bone 12 until the pairs of magnets 16 contact the fractured bone 12 in fixed positions with respect to the fractured bone 12 and one another and resulting from the attractive magnetic forces of the pairs of magnets 16.

The separation assembly 20 may be removed to the extent permitted by the connecting members 18 to a location away from the fracture to facilitate further treatment of the fractured bone 12. In some implementations, the magnets 16 may be joined with the connecting members 18 with removable clasps which enable the separation assembly 20 and connecting members 18 to be removed from the location of the fracture following the appropriate placement of the magnets 16 upon the fractured bone 12.

The separation assembly 20 and connecting members 18 may be re-attached to the magnets 16 to assist with removal of the magnets 16 from the fractured bone 12 and to assist with separation of the magnets 16 from one another during removal of the magnets 16 from the fractured bone 12 in one embodiment. In other embodiments, the pairs of magnets 16 are not removed from the patient and may remain attached to the fractured bone 12 to facilitate healing of the fractured bone 12.

Different sizes and configurations of the separation assembly 20 may be provided and used depending upon the size of fractured bone 12 of the patient to be treated. For example, different separation assemblies 20 may include more or less numbers of pairs of magnets 16 and which may be spaced at different distances from one another in different configurations.

In one aspect, the above-described fractured bone treatment assembly 10 facilitates treatment of fractured bones 12 by providing alignment and/or securely maintaining the alignment of a fractured bone 12 and allowing the surgeon to have greater access to the fractured bone 12 compared with some conventional treatment methods which may use mechanical clamps, etc. which may interfere to a greater degree with the surgeon's access to the fractured bone 12.

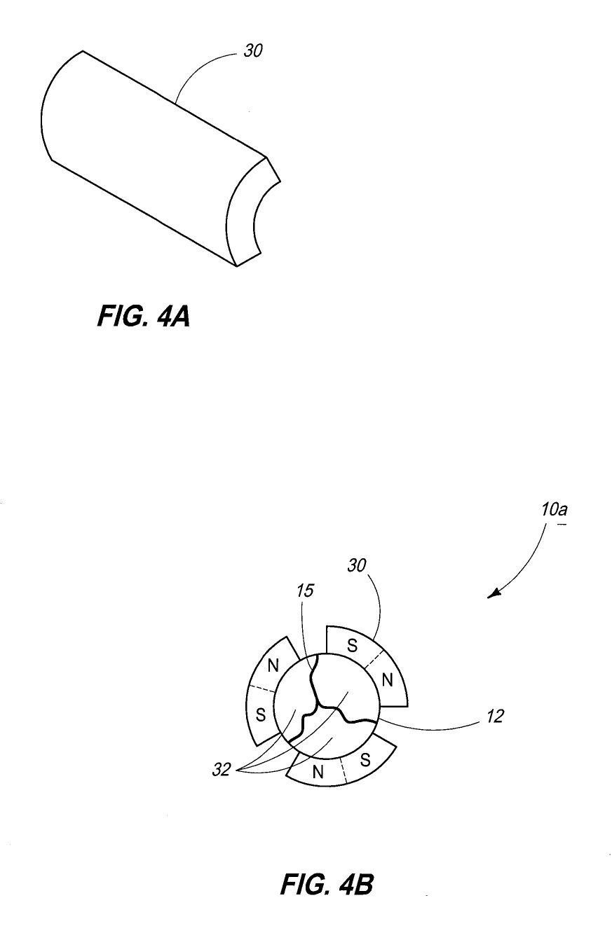

Referring to FIGS. 4A-4B, another configuration of a fractured bone treatment assembly 10a is described. A plurality of magnets 30 (permanent or electromagnetic) may be provided which are shaped to treat different sizes and types of bones. For example, the magnets 30 may have different lengths, widths, radii, etc. and may provide different magnetic forces and which may be designed to treat respective different fractured bones 12.

In one example treatment method, an incision is made in the appendage at a location proximate to the fracture and the soft tissue may be removed from the fractured bone 12 at the location of the fracture to expose the fractured bone 12. A plurality of magnets 30 which are shaped to mate with an exterior surface of the fractured bone 12 may be applied to the exterior surface of the fractured bone 12 to reduce the fracture by increasing alignment and/or maintaining proper alignment of the fractured bone (which may have been aligned by previous reduction).

The plurality of magnets 30 may be provided at substantially the same longitudinal position of the fractured bone at the location of the fracture in the fractured bone 12. In one embodiment, the magnets 30 may be positioned at different circumferential portions of the fractured bone 12 proximate to the fracture which may include a plurality of bone pieces or fragments 32. The treatment assembly 10a may be utilized with different types of bone fractures including maintaining proper anatomical alignment of bone fragments of comminuted fractures during treatment and healing in one embodiment. In the illustrated example, the magnets 30 are positioned to contact the fractured bone 12 at the different circumferential positions about the fractured bone 12 at approximately 45 degrees, 165 degrees and 285 degrees.

As shown in the example of FIG. 4B, the magnets 30 may be positioned such that the opposite poles of the magnets 30 are placed adjacent to one another to create attractive magnetic forces between the magnets 30 which operate to maintain the magnets 30 in a fixed position with respect to one another and the fractured bone 12 and to clamp and maintain the bone pieces or fragments 32 in fixed positions and in alignment with respect to one another.

The soft tissue may be placed about the fractured bone 12 and the magnets 30 and the incision may be closed following appropriate placement of the magnets 30 about the fractured bone 12 to maintain the alignment of the fractured bone 12. The attractive magnetic forces of the magnets 30 may be used to maintain the proper anatomical alignment of the fractured bone 12 for subsequent healing of the bone, such as during tissue growth. The magnets 30 may or may not be removed from the fractured bone 12 of the patient following the usage of the magnets 30 to heal the fracture. In other embodiments, the magnets 30 may be configured to encircle more or less portions of the fractured bone 12, for example, using more or less numbers of magnets 30 and/or magnets 30 of different sizes. In one embodiment, the magnets 30 may encircle a majority or substantially an entirety of the fracture 15 of the fractured bone 12.

Referring to FIG. 5, a fractured bone treatment assembly 10b is described according to an additional embodiment of the disclosure. Treatment assembly 10b includes a carrier apparatus 50 which is configured to provide and maintain one or more magnets in appropriate locations for treatment of a fractured bone 12.

In the illustrated example, the carrier apparatus 50 (e.g., a sleeve in one example) is wrapped about an appendage 51 of a patient which includes a fractured bone 12. In one embodiment, the carrier apparatus 50 includes a fastener 52, such as one or more hook-and-loop fastening regions, which permits the carrier apparatus 50 to be securely wrapped about the appendage 51 at a longitudinal location which is proximate to the fracture 15 in the bone 12 and which provides a plurality of magnets 54 (e.g., permanent or electromagnetic) adjacent to an exterior of the fractured bone 12.

The magnets 54 are provided in an arrangement wherein the opposing poles of adjacent magnets 54 are adjacent to one another providing an attractive magnetic force between the plurality of magnets 54. The attractive magnetic forces operate to improve alignment of the fractured bone 12 and/or maintain a previously aligned fractured bone 12 in alignment for healing when the carrier apparatus 50 is positioned about the appendage 51.

Different sizes of carrier apparatus 50 and magnets 54 may be used to treat different types of fractures as well as different sizes of bones. The fractured bone treatment assembly 10b may be utilized to align relatively minor fractures and/or stabilize fractured bones 12 which are in alignment for healing in different implementations. The carrier apparatus 50 may be provided about the appendage 51 of the patient to provide the fractured bone 12 in proper anatomical alignment for a period of time sufficient for the fractured bone 12 to heal in one treatment method.

The arrangement of the fractured bone treatment assembly 10b of FIG. 5 may be re-used as contrasted with conventional plaster casts or splints. In addition, the carrier apparatus 50 containing the magnets 54 may be quickly placed around the injured appendage 51 of the patient compared with lengths of time needed to form a conventional plaster cast or splint.

The different embodiments disclosed above may be applicable to treat different types of fractures, different types of fractured bone and different patients in different situations. Following the in-take of a patient with a fractured bone, it is determined weather surgery is needed to treat the fracture. If surgery is not needed, one embodiment provides wrapping of a carrier apparatus which includes magnets as discussed above around the appendage or limb of the individual which includes the fracture. The magnets may be selected and arranged within the carrier apparatus to provide an appropriate attractive magnetic force proximate to the fracture for treating the fracture. For example, the attractive magnetic force may provide alignment of the fracture bone including reduction of the fracture and/or be utilized to maintain pieces of the fracture in proper anatomical alignment for healing including growth of bone tissue.

If surgery is necessary, the fracture in the bone may be exposed by making an incision and retracting the soft tissue around the fracture. In one method, the fracture may be reduced, and following reduction, the magnets may be utilized to maintain or stabilize the fractured bone in proper anatomical alignment to permit one or more additional fixing devices, such as screws or plates, to be attached to the fractured bone for healing including growth of bone tissue. The magnets may be removed from the fractured bone after the fixing devices are attached or may remain in place within the patient to assist with stabilization of a fracture.

In another method, the magnets may be utilized to provide reduction of the fracture. For example, the magnets may be used to increase the alignment of the fractured bone during surgery. The magnets may also be used to maintain alignment of the fractured bone and stabilize the fractured bone with or without additional fixing devices following reduction of the fracture and during healing of the fracture, for example, including growth of bone tissue.

Different embodiments of the disclosure described above provide different methods for treating fractured bones using attractive magnetic forces. In addition to providing attractive magnetic forces to provide alignment and stabilization of fractured bones, magnets may also assist with bone tissue growth during the healing process. For example, the magnets may enhance the growth of bone tissue compared with arrangements where magnets are not utilized to treat bone fractures.

In addition, the magnets and/or treatment methods which are utilized may be selected according to the type of fractured bone and the type of fracture as well as other characteristics such as the age and sex of the individual being treated. For example, magnets of different sizes, shapes, and/or attractive powers may be utilized in different methods to provide appropriate treatment for different types of fractures, bones and patients.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

Further, aspects herein have been presented for guidance in construction and/or operation of illustrative embodiments of the disclosure. Applicant(s) hereof consider these described illustrative embodiments to also include, disclose and describe further inventive aspects in addition to those explicitly disclosed. For example, the additional inventive aspects may include less, more and/or alternative features than those described in the illustrative embodiments. In more specific examples, Applicants consider the disclosure to include, disclose and describe methods which include less, more and/or alternative steps than those methods explicitly disclosed as well as apparatus which includes less, more and/or alternative structure than the explicitly disclosed structure.

What is claimed is:

1. A fractured bone treatment method comprising:
generating an attractive magnetic force proximate to a fracture in a fractured bone; and
maintaining pieces of the fractured bone in proper anatomical positions with respect to one another using the attractive magnetic force.

2. The method of claim 1 further comprising aligning pieces of the fractured bone with respect to one another, and the maintaining comprises maintaining after the aligning.

3. The method of claim 1 further comprising attaching a fixing device to the fractured bone during the maintaining of the positions of the pieces of the fractured bone in the proper anatomical position with respect to one another using the attractive force.

4. The method of claim 1 further comprising placing a carrier apparatus which includes the magnets about an appendage which includes the fractured bone.

5. The method of claim 1 further comprising, before the maintaining, reducing non-alignment of the pieces of the fractured bone using the attractive magnetic force.

6. The method of claim 1 wherein the generating comprises generating the attractive magnetic force between a plurality of magnets which are positioned on different sides of the fracture of the fractured bone.

7. The method of claim 6 wherein the generating comprises generating the attractive force between a plurality of pairs of the magnets which are positioned proximate to the fracture in the fractured bone, and wherein opposite poles of the magnets of an individual one of the pairs are positioned adjacent to the fractured bone.

8. The method of claim 1 wherein the generating comprises generating the attractive force between a plurality of magnets which are positioned adjacent to different circumferential portions of the fractured bone proximate to the fracture, and wherein opposing poles of adjacent ones of the magnets are positioned adjacent to one another.

9. The method of claim 1 wherein the generating comprises generating using a plurality of magnets which are in contact with an exterior surface of the fractured bone.

10. The method of claim 1 further comprising enhancing tissue growth of the fractured bone using the magnets compared with an amount tissue growth of the fractured bone which occurs in an absence of the magnets.

11. A fractured bone treatment method comprising:
providing a plurality of magnets adjacent to an exterior surface of a fractured bone at a location which is proximate to a fracture in the fractured bone, wherein the providing comprises providing the magnets in an arrangement wherein an attractive magnetic force is generated using the plurality of magnets; and
aligning the fractured bone using the attractive magnetic force generated the plurality of magnets.

12. The method of claim 11 further comprising attaching at least one fixing device to the fractured bone while the magnets are placed adjacent to the exterior surface of the fractured bone to generate the attractive magnetic force.

13. The method of claim 11 wherein the aligning comprises maintaining proper anatomical alignment of the fractured bone using the attractive magnetic force during healing of the fractured bone.

14. The method of claim 11 wherein the providing comprises initially providing the magnets adjacent to the fractured bone which is in a non-aligned state, and wherein the aligning comprises reducing the non-alignment of the fractured bone using the attractive magnetic force.

15. The method of claim 14 further comprising controlling the attractive magnetic force including increasing the attractive magnetic force during the reducing.

16. The method of claim 11 wherein the aligning comprises maintaining a plurality of pieces of the fractured bone in proper anatomical alignment with respect to one another.

17. The method of claim 11 further comprising separating a plurality of magnets from one another during the providing using a separation assembly.

18. The method of claim 11 further comprising controlling the attractive magnetic force of the magnets during the providing and the aligning.

19. The method of claim 18 wherein the controlling comprises increasing the attractive magnetic force after the providing and during the aligning.

20. The method of claim 11 wherein the providing comprises providing a pair of the magnets adjacent to opposing sides of the fractured bone in the arrangement wherein opposite poles of the magnets of the pair are provided adjacent to the exterior surface of the fractured bone to generate the attractive magnetic force.

21. The method of claim 11 wherein the providing comprises providing a plurality of pairs of the magnets adjacent to different sides of the fractured bone and at a plurality of different longitudinal positions of the fractured bone with opposing poles of the magnets of an individual one of the pairs adjacent to the exterior surface of the fractured bone.

22. The method of claim 11 wherein the providing comprises providing more than two of the magnets at different circumferential locations about the fracture in the fractured bone.

23. The method of claim 22 wherein the providing comprises providing opposing poles of adjacent ones of the magnets adjacent to one another.

24. The method of claim 11 wherein the providing comprises providing a carrier apparatus which includes the magnets about an appendage which includes the fractured bone.

25. The method of claim 11 wherein the providing comprises providing the magnets in contact with the exterior surface of the fractured bone.

26. A fractured bone treatment assembly comprising:
a plurality of pairs of magnets, wherein the magnets of an individual one of the pairs are coupled with one another using a connecting member which is configured to permit the magnets of the individual one of the pairs to be placed adjacent to opposing exterior surfaces of a fractured bone in an arrangement wherein an attractive magnetic force is generated by the magnets which maintains the fractured bone in proper anatomical alignment; and
a separation assembly configured to provide separation of the magnets of the pairs from one another during placement of the magnets adjacent to the opposing exterior surfaces of the fractured bone.

27. The assembly of claim 26 wherein the magnets of the pairs of magnets contact the exterior surfaces of the fractured bone.

28. The assembly of claim 26 wherein opposing poles of the magnets of an individual one of the pairs are provided adjacent to the exterior surfaces of the fractured bone.

29. The assembly of claim 26 wherein the pairs of magnets are positioned adjacent to different longitudinal positions of the fractured bone.
